(19) United States
(12) Patent Application Publication
(10) Pub. No.: US 2011/0060316 A1
(43) Pub. Date: Mar. 10, 2011

(54) Tipped Ribbon Integrated Guidewire

(76) Inventor: Paul DiCarlo, Middleboro, MA (US)

(21) Appl. No.: 12/554,613

(22) Filed: Sep. 4, 2009

(51) Int. Cl. A61M 25/09; A61M 25/098

(52) U.S. Cl. 604/529; 604/528

(57) ABSTRACT

A guidewire having a longitudinal axis includes an inner ribbon coil wound in a first direction, an outer ribbon coil wound in a second direction opposite the first direction wherein the inner ribbon coil is coaxially arranged within the outer ribbon coil and configured to prevent the inner ribbon coil from interlocking with the outer ribbon coil, and a plurality of connection points along the longitudinal axis of the guidewire wherein the plurality of connection points affixes the inner ribbon coil to the outer ribbon coil.

### FIELD

The present disclosure relates generally to steerable guidewires, and more particularly, to the manufacture and use of a dual-ribboned integrated guidewire configured to be a highly effective alternative to traditional guidewires used in the medical field.

### BACKGROUND

Medical grade guidewires are well known in the field and are used for purposes of diagnosis and treatment in various body lumens. Typical guidewires, however, are prone to wiping when applying rotational force to one end of the guidewire, i.e., the proximal end, causing the distal end of the guidewire to lag in rotational movement. Depending on the particular application of the guidewire, this wiping quality is entirely undesirable and creates uncertainty in the movement of the guidewire as it travels within the body. It becomes even more important to navigate the guidewire with reliable consistency and accuracy through a body due to the anticipated tortuous paths the guidewire must navigate prior to reaching the treatment site. Accordingly, there is a need in the field for a medical-grade guidewire that is capable of eliminating the wiping problem and, thus, providing certainty and precision to the navigational placement of the guidewire.

### SUMMARY

One aspect of a guidewire is herein disclosed. A guidewire having a longitudinal axis includes an inner ribbon coil wound in a first direction, an outer ribbon coil wound in a second direction opposite the first direction wherein the inner ribbon coil is coaxially arranged within the outer ribbon coil and configured to prevent the inner ribbon coil from interlocking with the outer ribbon coil, and a plurality of connection points along the longitudinal axis of the guidewire wherein the plurality of connection points affixes the inner ribbon coil to the outer ribbon coil.

Another aspect of a guidewire is also disclosed. A medical guidewire having a longitudinal axis capable of entering a body lumen includes a flat inner ribbon coil wound in a first direction having a distal end and a proximal end, a flat outer ribbon coil wound in a direction opposite to the first direction wherein the flat inner ribbon coil and the outer ribbon coil are coaxially arranged relative to one another, a bulbous tip operatively connected to the flat inner and flat outer ribbon coils at the distal end configured to guide the guidewire through the body lumen, and a plurality of connection points along the longitudinal axis of the guidewire wherein the plurality of connection points affixes the flat ribbon coils together.

These, as well as other objects, features, and benefits will now become clear from a review of the following detailed description of illustrative embodiments and the accompanying drawings.

### BRIEF DESCRIPTION OF THE DRAWINGS

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely:

FIG. 1 is a lateral view of a dual-ribboned integrated guidewire.

FIG. 2 is a lateral cross-sectional view of the dual-ribboned portion of an integrated guidewire.

FIG. 3 is a lateral cross-sectional view of the tip portion a dual-ribboned integrated guidewire.

FIG. 4 is an elevated cross sectional view of the tip portion of a dual-ribboned integrated guidewire.

FIG. 5 is an elevated lateral view of the tip portion of a dual-ribboned integrated guidewire with a spacer.

FIG. 6 is an elevated lateral view of the tip portion of a dual-ribboned integrated guidewire without a spacer.

### DETAILED DESCRIPTION

Each of the additional features and teachings disclosed below can be utilized separately or in conjunction with other features and teachings to provide a medical grade guidewire for use in body lumens. Representative examples which examples utilize many of these additional features and teachings both separately and in combination, will now be described in further detail with reference to the attached drawings. This detailed description is merely intended to teach a person of skill in the art further details for practicing preferred aspects of the present teachings and is not intended to limit the scope of the disclosure. Therefore, combinations of features and steps disclosed in the following detail description may not be necessary to practice the teachings in the broadest sense, and are instead taught merely to particularly describe representative examples of the present teachings.

Moreover, the various features of the representative examples and the dependent claims may be combined in ways that are not specifically and explicitly enumerated in order to provide additional useful embodiments of the present teachings. In addition, it is expressly noted that all features disclosed in the description and/or the claims are intended to be disclosed separately and independently from each other for the purpose of original disclosure, as well as for the purpose of restricting the claimed subject matter independent of the compositions of the features in the embodiments and/or the claims. It is also expressly noted that all value ranges or indications of groups of entities disclose every possible intermediate value or intermediate entity for the purpose of original disclosure, as well as for the purpose of restricting the claimed subject matter.

The detailed description set forth below in connection with the appended drawings are intended as a description of various embodiments and is not intended to represent the only embodiments which may be practiced. The detailed description includes specific details for providing a thorough understanding. It will be apparent to those skilled in the art, however, that the embodiment may be practiced without these specific details.

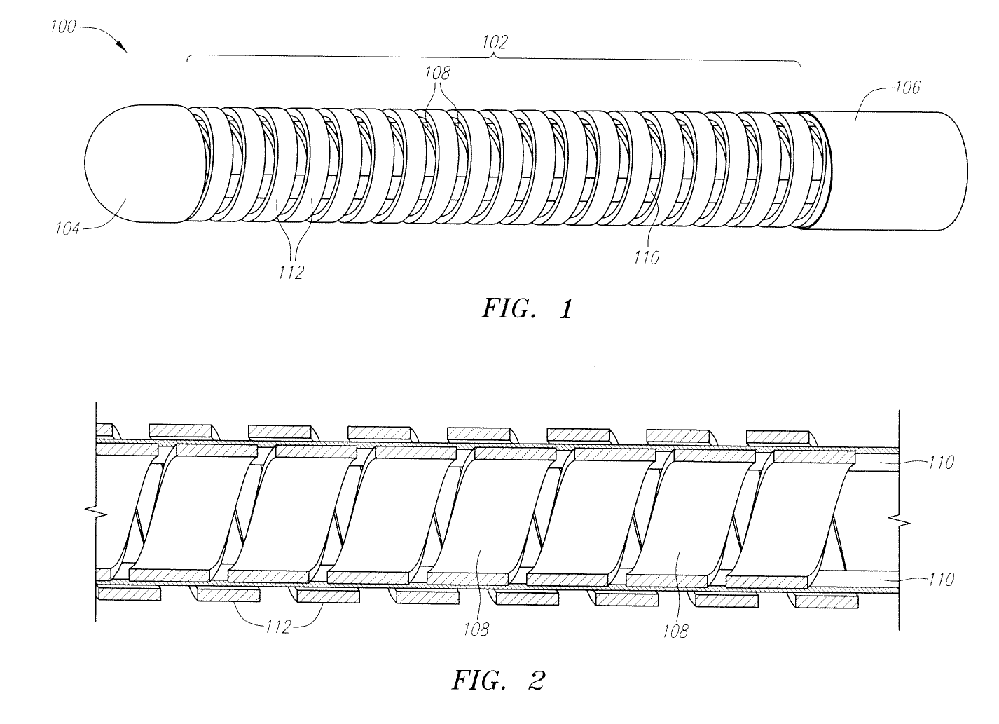

FIG. 1 is a lateral view of a dual-ribboned integrated guidewire 100. The guidewire 100 is generally comprised of three components: the tip 104, the ribboned portion 102, and the standard wire body 106. Although the components 102, 104, and 106 are herein separately described, one of ordinary skill may appreciate that for all practical purposes the guidewire 100 consists of one uniform piece appropriate for navigating a variety of body lumens. As shown, a flat outer ribbon coil 112 is wound in one direction to form a substantially cylindrical body. Also contemplated herein is the use of additional geometries for the outer ribbon coil 112. For example, one of ordinary skill may choose to use a round ribbon coil, hexagonal ribbon coil, or any other myriad of geometries (not shown) without deviating from the teachings of this specification. Such choices would merely be design decisions based on the anticipated use of the guidewire 100.

An inner coil 108 is manufactured to be wound in the exact opposition direction of the outer coil 112. The torsional strength of the outer coil 112 and the inner coil 108 must be substantially equated so that the coils may counteract each other's rotational force. This counteraction of forces results in the ability to maintain the guidewire 100 in equilibrium when at rest—that is, the guidewire 100 does not rotate in either direction without any external force applied to it. Once an external torsional force is applied to the guidewire 100 at either end, that rotational force is instantly and seamlessly transmitted to the other end of the guidewire 100. This arrangement prevents wiping or rotational slack that would otherwise be present absent this arrangement. The placement and navigational ability of the guidewire 100 is critically important when placing the guidewire 100 in a body lumen. Thus, it is a salient point of novelty that the guidewire 100 disclosed herein provides a medical practitioner with the ability to use this device with unprecedented certainty and control.

Once manufactured, the inner coil 108 may be placed or otherwise inserted into the hollow cylindrical structure of the outer coil 112. The inner coil 108 and the outer coil 112 are thus placed in coaxial arrangement in relation to one another. The inner coil 108 is affixed to the outer coil 112, or vice versa, by applying a series of spot welds or, alternatively, laser beam welds at various spaced positions along the entire shaft of the coaxial arrangement. Depending on the coil geometries, one of ordinary skill could reasonably deduce that a spot weld may be necessary every two through ten turns wherein each turn is one loop of wire. Likewise, instead of welding the inner and outer coils to each other, alternative embodiments can use an arrangement of adhesives to keep the coils affixed to one another, e.g., any medical grade epoxy.

Because of the novel arrangement described herein, the guidewire 100 assembly is able to act as a slotted tube but without the costs and drawbacks associated with the cutting and deburring processes. As is generally known in the art, the combination of two coils immediately adjacent to one another may cause one coil to ‘catch’ or engage on the other coil as they move in relation to each other, i.e., galling. This potential problem may be addressed by using a spacer 110. The spacer 110 lies directly between the outer coil 112 and the inner coil 108 and prevents the coils from engaging each other. In order to lessen the impact that any friction may have, the spacer 110 may be made from a lubricious hydrophilic material. Moreover, although the spacer 110 is depicted as consisting of a unitary strip running along the length of the ribboned portion 102 of the guidewire 100, the spacer 110 may also be a flexible cylindrical structure that extends substantially about the inner coil 108. Alternatively, the spacer 110 may also consist of a series of strips, operatively connected to each other or in independent arrangement that substantially carries out the same function as the spacer 110.

FIG. 2 is a lateral cross-sectional view of the dual-ribboned portion 102 of an integrated guidewire 100. The cross-section illustrates the internal structure of the inner coil 108 in relation to the outer coil 112 and the spacer 110. In the embodiment depicted by FIG. 2, there are two spacers 110, both substantially at opposite ends of the inner coil 108 circumference. Since the guidewire 100 is specifically manufactured for use in body lumens, it is important to note that the inner coil 108 and outer coil 112 should be made of medical grade material, preferably Nitinol or other elastic or superelastic alloy.

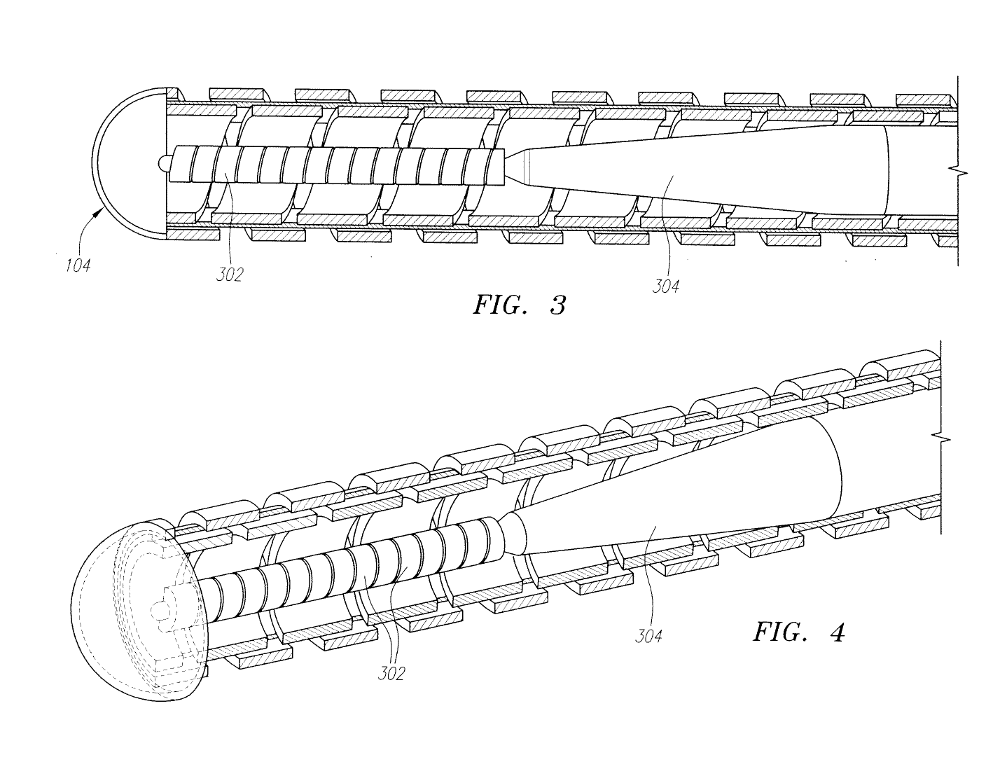

FIG. 3 is a lateral cross-sectional view of the tip 104 portion of a dual-ribboned integrated guidewire 100. The tip 104 may be any shape having atraumatic qualities, e.g., rounded, coned, or any other shape that facilitates navigation through a body lumen without damaging surrounding tissues. The bulbous tip 104 is described in the illustrated embodiment but these alternative tip structures could just as easily substitute for same. The tip 104 is coupled to at least the inner coil 108 and the outer coil 112 so as to stabilize torsional forces of both. This stabilization may be accomplished by welding or otherwise fusing the tip 104 to at least either of the inner or outer coil.

In further coaxial arrangement, the depicted embodiment illustrates the configuration of the guidewire 100 with a radiopaque element 302 at the distal end of the guidewire and substantially enveloped by the hollow cylindrical structure formed by the dual-ribboned portion 102 of the guidewire 100. Typically, although not necessarily a limitation, medical grade guidewires include a radiopaque 302 element at a distal end to make its presence visible when exposed to x-ray, cat scan, or fluoroscopy procedures. The radiopaque element 302 is held in position at the distal end by the tip 104, which has a receiving portion at the center. The proximal end of the radiopaque element 302 is held central to the cylindrical body by a standard core wire 304. In some embodiments, the core wire 304 may become the standard wire body 106 at the proximal end. In others, the core wire 304 runs substantially along and within the ribboned structure 102 and is operatively coupled to the standard wire body 106.

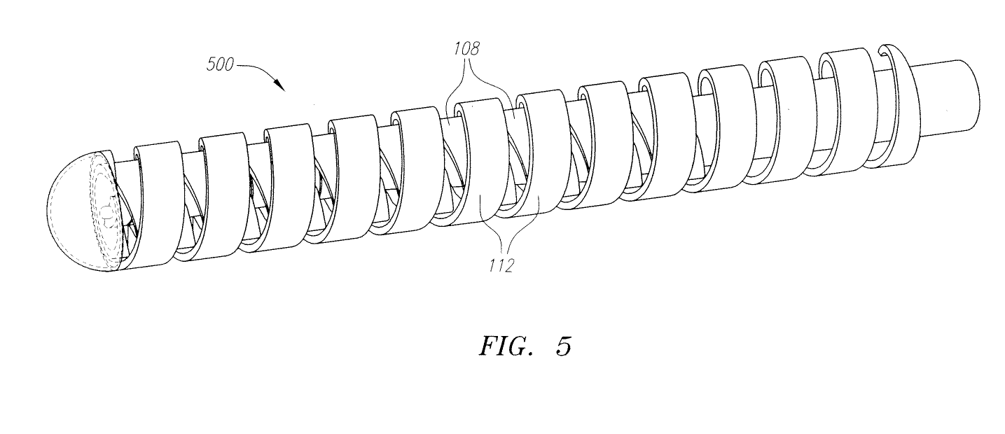

FIG. 4 is an elevated cross sectional view of the tip portion of a dual-ribboned integrated guidewire. The components previously described in connection with FIG. 3 are identical to those referenced by FIG. 4. Accordingly, the prior discussion equally applies here. FIG. 4 is included to give a practitioner having ordinary skill an additional perspective of the disclosed embodiment.

FIG. 5 is an elevated lateral view of the tip portion of a dual-ribboned integrated guidewire without a spacer. An alternative embodiment not requiring the use of a spacer 110 can address the previously discussed issue relating to galling by implementing a lubricious coating along the inside circumference of the outer coil 112, the outer circumference of the inner coil 108, or both. For example, by introducing a low temperature polymer to the dual ribboned structure, either as a strip spacer 110 or a sleeved spacer (not shown), one of ordinary skill could apply heat and have the polymer plastically deform about the ribbon under slight pressure. In yet a different embodiment, rather than using a temperature controlled polymer, a diamond-like carbon coating (DLC) may be applied to the friction areas between the inner and outer coils, thus, obviating the need for any spacer due to the inherent hardness and minimal galling. Likewise, TiNi (titanium nitride) may be applied to the outer circumference of the inner ribbon coil 108 or the inner circumference of the outer ribbon coil 112, or both, so as to reduce the friction of coefficient between the coils.

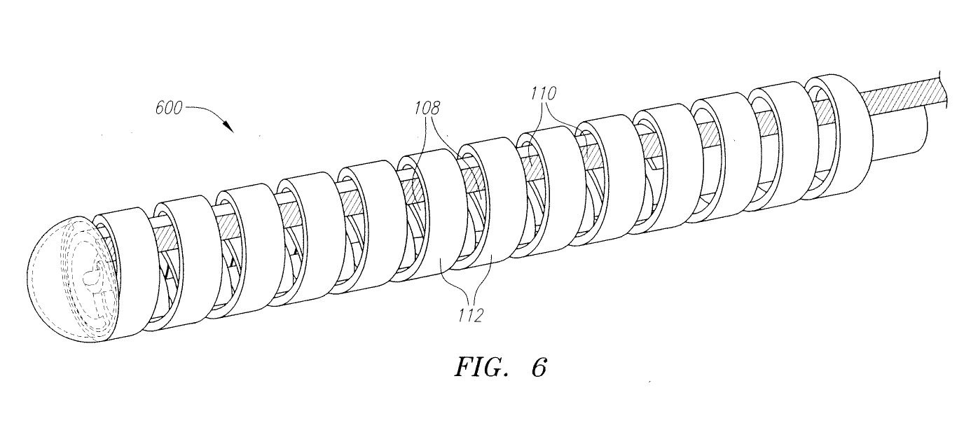

FIG. 6 is an elevated lateral view of the tip portion of a dual-ribboned integrated guidewire with a spacer 110. As previously disclosed, the use of a spacer 110 is not necessary to carry out the disclosed teachings and is only included as an illustrative embodiment that takes an opportunity to implement a spacer structure. Typically, the spacer 110 material will consist of TEFLON or similar highly viscous material. Regardless of the actual inerts of the guidewire 100, one of ordinary skill may appreciate that by applying a super hydrophobic coating to the external, exposed portion of the guidewire 100, the guidewire 100 will be easier to navigate body lumens while minimizing the potential for damaging the surrounding tissues.

While the specification describes particular embodiments of the present invention, those of ordinary skill can devise variations of the present invention without departing from the inventive concept. Also, the previous description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but is to be accorded the full scope consistent with the language of the claims, wherein reference to an element in the singular is not intended to mean “one and only one” unless specifically so stated, but rather “one or more.”

Moreover, all structural and functional equivalents to the elements of the various embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

What is claimed is:

1. A guidewire having a longitudinal axis comprising:
an inner ribbon coil wound in a first direction;
an outer ribbon coil wound in a second direction opposite the first direction wherein the inner ribbon coil is coaxially arranged within the outer ribbon coil and configured to prevent the inner ribbon coil from interlocking with the outer ribbon coil; and
a plurality of connection points along the longitudinal axis of the guidewire wherein the plurality of connection points affixes the inner ribbon coil to the outer ribbon coil.

2. The guidewire of claim 1 further comprising a substantially rounded tip at a distal end of the guidewire configured to cap the outer ribbon coil or the inner ribbon coil.

3. The guidewire of claim 2 wherein the inner ribbon coil is substantially flat.

4. The guidewire of claim 3 wherein the outer ribbon coil is substantially flat.

5. The guidewire of claim 4 further comprising a spacer in between the inner ribbon coil and the outer ribbon coil.

6. The guidewire of claim 5 wherein the spacer is made of a polymer.

7. The guidewire of claim 6 wherein the polymer is a low temperature polymer that deforms about the inner and outer ribbon coils when heat is applied to the ribbons.

8. The guidewire of claim 5 wherein the spacer is made from a weldable material that may be welded to at least one of the ribbon coils.

9. The guidewire of claim 4 wherein the inner ribbon coil and the outer ribbon coil are laser welded at the plurality of connection points.

10. The guidewire of claim 4 wherein the inner ribbon coil and the outer ribbon coil are adhered to each other at the plurality of connection points.

11. The guidewire of claim 4 further comprising a hydrophilic coating residing between the outer ribbon coils.

12. The guidewire of claim 4 further comprising a radiopaque marker placed at the distal end of the guidewire.

13. A medical guidewire having a longitudinal axis capable of entering a body lumen comprising:
a flat inner ribbon coil wound in a first direction having a distal end and a proximal end;
a flat outer ribbon coil wound in a direction opposite to the first direction wherein the flat inner ribbon coil and the outer ribbon coil are coaxially arranged relative to one another;
a bulbous tip operatively connected to the flat inner or flat outer ribbon coils at the distal end configured to guide the guidewire through the body lumen; and
a plurality of connection points along the longitudinal axis of the guidewire wherein the plurality of connection points affixes the flat ribbon coils together.

14. The guidewire of claim 13 further comprising a spacer placed substantially between the flat inner ribbon coil and the flat outer ribbon coil.

15. The guidewire of claim 14 wherein the spacer is made of a polymer.

16. The guidewire of claim 15 wherein the polymer is a low temperature polymer that deforms about the inner and outer ribbon coils when heat is applied to the ribbon coils.

17. The guidewire of claim 14 wherein the spacer is made from a weldable material that may be welded to at least one of the ribbon coils.

18. The guidewire of claim 17 wherein the inner ribbon coil and the outer ribbon coil are laser welded at the plurality of connection points.

19. The guidewire of claim 14 wherein the inner ribbon coil and the outer ribbon coil are adhered to each other at the plurality of connection points.

20. The guidewire of claim 13 further comprising a hydrophilic coating residing between the outer ribbon coils.

21. The guidewire of claim 13 further comprising a radiopaque marker placed at the distal end of the guidewire in coaxial arrangement with the flat inner ribbon coil.
